(19) United States
(12) Patent Application Publication
(10) Pub. No.: US 2017/0165411 A1
(43) Pub. Date: Jun. 15, 2017

(54) Vaginal Hygiene Assembly

(71) Applicant: Barbara Peters, Carson, CA (US)

(72) Inventor: Barbara Peters, Carson, CA (US)

(21) Appl. No.: 14/963,941

(22) Filed: Dec. 9, 2015

(51) Int. Cl. A61M 3/02

(52) U.S. Cl. CPC A61M 3/0262; A61M 2210/1475

(57) ABSTRACT

A vaginal hygiene assembly for cleaning and disinfecting a vaginal canal includes a bulb that may contain a solution. The bulb has an elongated neck and the elongated neck may be inserted into a vaginal canal. The bulb is comprised of a resiliently compressible material. Thus, the bulb may be manipulated thereby facilitating the solution to be urged into the vaginal canal.

### BACKGROUND OF THE DISCLOSURE

Field of the Disclosure

The disclosure relates to hygiene devices and more particularly pertains to a new hygiene device for cleaning and disinfecting a vaginal canal.

### SUMMARY OF THE DISCLOSURE

An embodiment of the disclosure meets the needs presented above by generally comprising a bulb that may contain a solution. The bulb has an elongated neck and the elongated neck may be inserted into a vaginal canal. The bulb is comprised of a resiliently compressible material. Thus, the bulb may be manipulated thereby facilitating the solution to be urged into the vaginal canal.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a top perspective view of a vaginal hygiene assembly according to an embodiment of the disclosure.

FIG. 2 is a front view of an embodiment of the disclosure.

FIG. 3 is a bottom view of an embodiment of the disclosure.

FIG. 4 is a cross sectional view taken along line 4-4 of FIG. 3 of an embodiment of the disclosure.

FIG. 5 is a perspective in-use view of an embodiment of the disclosure.

### DESCRIPTION OF THE PREFERRED EMBODIMENT

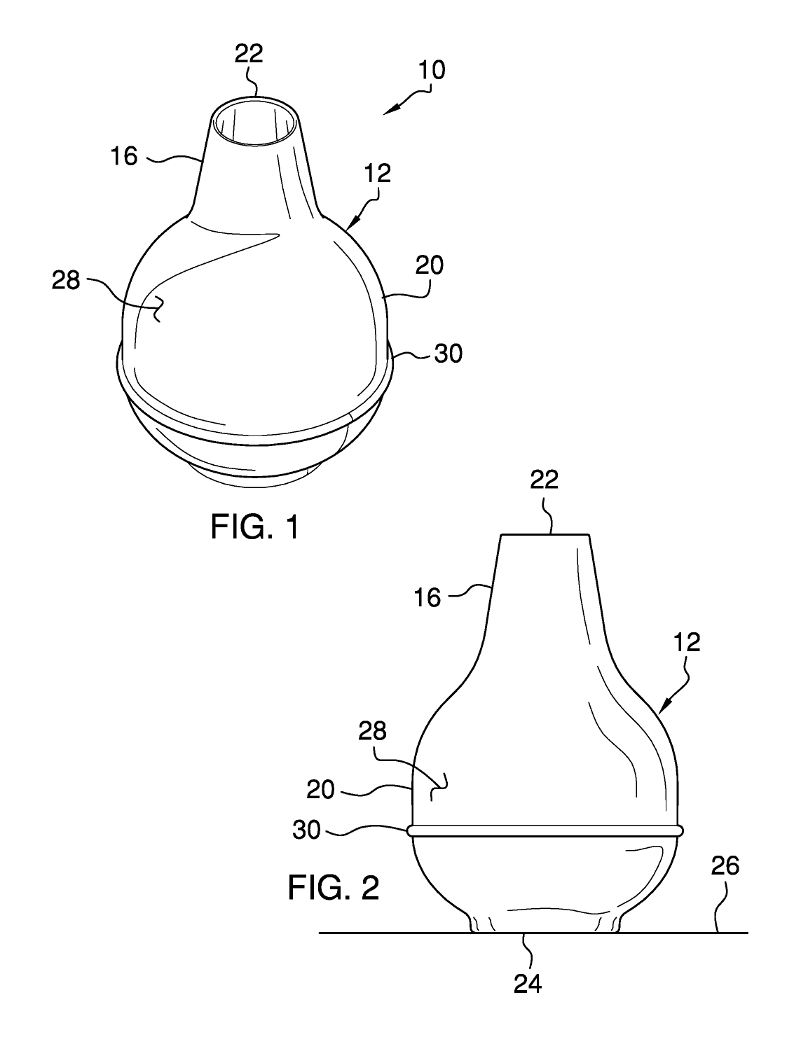

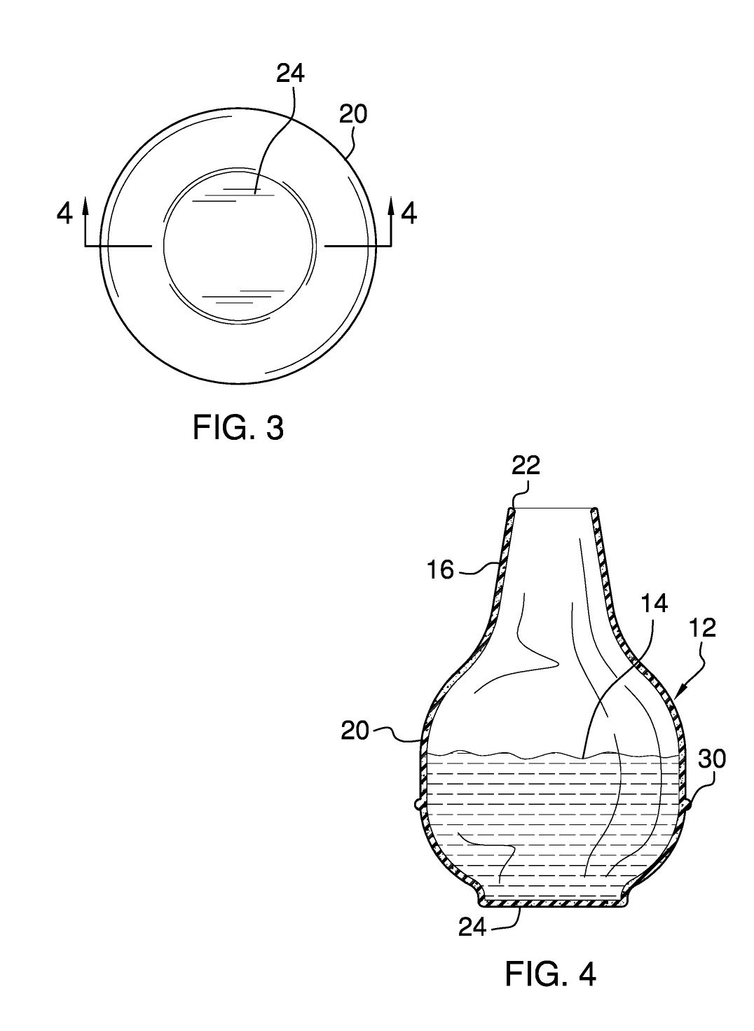

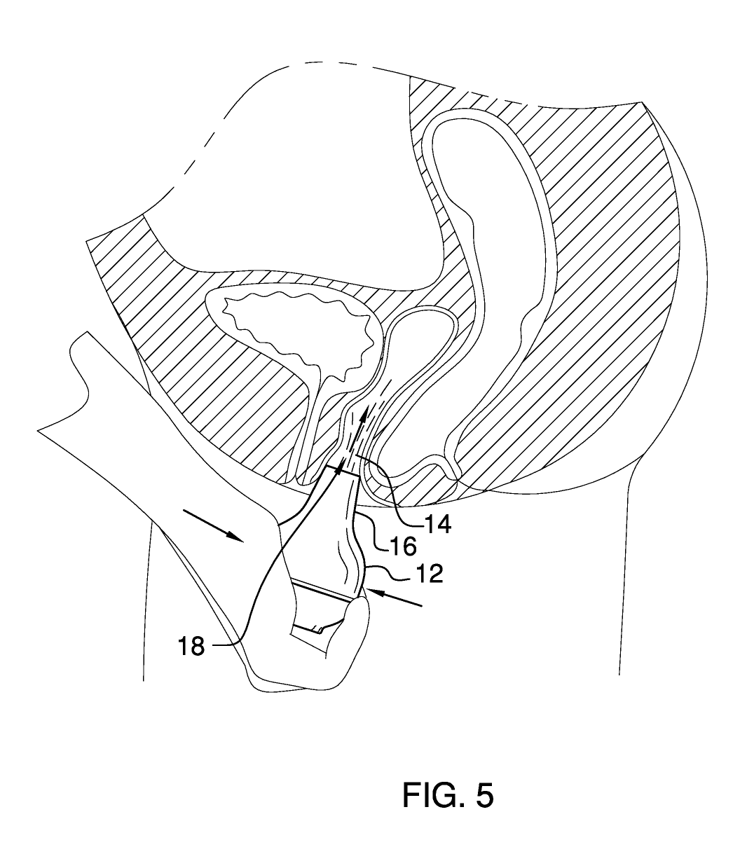

With reference now to the drawings, and in particular to FIGS. 1 through 5 thereof, a new hygiene device embodying the principles and concepts of an embodiment of the disclosure and generally designated by the reference numeral 10 will be described.

As best illustrated in FIGS. 1 through 5, the vaginal hygiene assembly 10 generally comprises a bulb 12 that may contain a solution 14. The solution 14 may comprise a mixture of vinegar and water or the like. Additionally, the solution 14 may comprise a non-toxic and anti-bacterial solution or the like. The bulb 12 has an elongated neck 16 and the elongated neck 16 may be inserted into a vaginal canal 18. The bulb 12 is comprised of a resiliently compressible material. Thus, the bulb 12 may be compressed thereby facilitating the solution 14 to be urged into the vaginal canal 18.

The bulb 12 has a reservoir portion 20 and the elongated neck 16 extends upwardly from the reservoir portion 20. The elongated neck 16 is substantially hollow and the elongated neck 16 has a distal end 22 with respect to the reservoir portion 20. The reservoir portion 20 is substantially hollow and the reservoir portion 20 contains the solution 14. The elongated neck 16 is in fluid communication with the reservoir portion 20. The distal end 22 is open to release the solution 14 from the reservoir portion 20 when the reservoir portion 20 is manipulated.

The elongated neck 16 tapers inwardly between the reservoir portion 20 and the distal end 22. Thus, the elongated neck 16 may comfortably enter the vaginal canal 18. The reservoir portion 20 has a bottom end 24. The bottom end 24 is flattened and the bottom end 24 may be positioned on a support surface 26. Thus, the bulb 12 may remain upright on the support surface 26. The support surface 26 may comprise a table or the like. The elongated neck 16 may have a length ranging between five cm and ten cm.

The elongated neck 16 is oriented such that a line extends between the distal end 22 and the bottom end 24. The reservoir portion 20 has an outer surface 28. The outer surface 28 has a ring 30 extending around an entire circumference of the reservoir portion 20. Thus, the ring 30 enhances gripping the reservoir portion 20. The ring 30 may be centrally positioned between the bottom end 24 and the elongated neck 16.

In use, the solution 14 is poured through the distal end 22 to fill the reservoir portion 20. The elongated neck 16 is inserted into the vaginal canal 18. The reservoir portion 20 is compressed and the solution 14 is urged into the vaginal canal 18. The solution 14 cleans and disinfects the vaginal canal 18. The elongated neck 16 is removed from the vaginal canal 18 when the reservoir portion 20 is emptied.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A vaginal hygiene assembly comprising:
a bulb being configured to contain a solution, said bulb having an elongated neck wherein said elongated neck is configured to be inserted into a vaginal canal, said bulb being comprised of a resiliently compressible material wherein said bulb is configured to be manipulated thereby facilitating the solution to be urged into the vaginal canal, said bulb having a reservoir portion, said elongated neck extending upwardly from said reservoir portion, said elongated neck being substantially hollow, said elongated neck having a distal end with respect to said reservoir portion, said reservoir portion being substantially hollow wherein said reservoir portion is configured to contain the solution, said elongated neck being in fluid communication with said reservoir portion, said reservoir portion has an outer surface, said outer surface having a ring extending around an entire circumference of said reservoir portion wherein said ring is configured to enhance gripping said reservoir portion, said outer surface of said reservoir portion being smooth and hemi-spherical extending from a base of said elongated neck to said ring.

2. (canceled)

3. The assembly according to claim 1, wherein said distal end is open wherein said distal end is configured to release the solution from said reservoir portion when said reservoir portion is manipulated, said elongated neck tapering inwardly between said reservoir portion and said distal end wherein said elongated neck is configured to comfortably enter the vaginal canal.

4. The assembly according to claim 1, wherein
said reservoir portion has a bottom end, said bottom end being flattened wherein said bottom end is configured to be positioned on a support surface thereby facilitating said bulb to remain upright on the support surface.

5. (canceled)

6. A vaginal hygiene assembly comprising:
a bulb being configured to contain a solution, said bulb having an elongated neck wherein said elongated neck is configured to be inserted into a vaginal canal, said bulb being comprised of a resiliently compressible material wherein said bulb is configured to be manipulated thereby facilitating the solution to be urged into the vaginal canal, said bulb having a reservoir portion, said elongated neck extending upwardly from said reservoir portion, said elongated neck being substantially hollow, said elongated neck having a distal end with respect to said reservoir portion, said reservoir portion being substantially hollow wherein said reservoir portion is configured to contain the solution, said elongated neck being in fluid communication with said reservoir portion, said distal end being open wherein said distal end is configured to release the solution from said reservoir portion when said reservoir portion is manipulated, said elongated neck tapering inwardly between said reservoir portion and said distal end wherein said elongated neck is configured to comfortably enter the vaginal canal, said reservoir portion having a bottom end, said bottom end being flattened wherein said bottom end is configured to be positioned on a support surface thereby facilitating said bulb to remain upright on the support surface, said reservoir portion having an outer surface, said outer surface having a ring extending around an entire circumference of said reservoir portion wherein said ring is configured to enhance gripping said reservoir portion, said outer surface of said reservoir portion being smooth and hemi-spherical extending from a base of said elongated neck to said ring.
